(19) United States
(12) Patent Application Publication
(10) Pub. No.: US 2019/0269703 A1
(43) Pub. Date: Sep. 5, 2019

(54) MATERIALS AND METHODS FOR TREATING CHRONIC COUGH

(71) Applicant: Mayo Foundation for Medical Education and Research, Rochester, MN (US)

(72) Inventors: Kaiser G. Lim, Mendota Heights, MN (US); Roger A. Warndahl, Rochester, MN (US)

(73) Assignee: Mayo Foundation for Medical Education and Research, Rochester, MN (US)

(21) Appl. No.: 16/302,205

(22) PCT Filed: May 16, 2017
(86) PCT No.: PCT/US2017/032841

§ 371 Date: Nov. 16, 2018

Related U.S. Application Data
(60) Provisional application No. 62/337,601, filed on May 17, 2016.

(51) Int. Cl. A61K 31/58; A61K 31/46; A61K 31/138; A61K 9/19; A61K 9/00; A61K 9/08; A61M 15/08; A61M 11/00

(52) U.S. Cl. CPC A61K 31/58; A61K 31/46; A61K 31/138; A61M 11/00; A61K 9/0043; A61K 9/08; A61M 15/08; A61K 9/19

(57) ABSTRACT

This document relates to methods and materials for treating chronic cough, nasal congestion, and/or one or more conditions causing chronic cough and/or nasal congestion (e.g., posterior rhinorrhea, also known as post-nasal drip (PND), and/or rhinitis). For example, this document relates to compositions containing a steroid (e.g., mometasone furoate), an anticholinergic (e.g., ipratropium bromide), and an antihistamine (e.g., diphenhydramine) as well as methods of using such compositions to reduce or eliminate one or more conditions causing chronic cough (e.g., PND and/or rhinitis).

### CROSS-REFERENCE TO RELATED APPLICATIONS

This application claims priority to U.S. Provisional Application Ser. No. 62/337,601, filed on May 17, 2016. This disclosure of the prior application is considered part of (and is incorporated by reference in) the disclosure of this application.

### BACKGROUND

### 1. Technical Field

This document relates to methods and materials for treating chronic cough, nasal congestion, and/or one or more conditions causing chronic cough (e.g., posterior rhinorrhea, also known as post-nasal drip (PND), and/or rhinitis). For example, this document relates to compositions containing a steroid (e.g., mometasone furoate), an anticholinergic (e.g., ipratropium bromide), and an antihistamine (e.g., diphenhydramine) as well as methods of using such compositions to reduce or eliminate nasal congestion and/or one or more conditions causing chronic cough (e.g., PND and/or rhinitis).

### 2. Background Information

Rhinitis and PND account for 60% or more of chronic cough. The American College of Chest Physicians (ACCP) 2006 Cough Guidelines has included rhinitis and postnasal drip into an umbrella syndrome called Upper Airway Cough Syndrome (UACS). The ACCP recommends an empiric trial of oral decongestant and a first generation antihistamine for PND. However, many patients fail this regimen and continue to have cough due to PND. In addition, many patients cannot tolerate this regimen because of sedation from the first generation antihistamines. There are medical concerns over the use of decongestants especially in older individuals, and two oral decongestants have been withdrawn from the market over time because of association with subarachnoid bleeding.

### SUMMARY

This document relates to methods and materials for treating chronic cough, nasal congestion, and/or one or more conditions causing chronic cough (e.g., PND and/or rhinitis). For example, this document relates to compositions containing a steroid (e.g., mometasone furoate), an anticholinergic (e.g., ipratropium bromide), and an antihistamine (e.g., diphenhydramine) as well as methods of using such compositions to reduce or eliminate nasal congestion and/or one or more conditions causing chronic cough (e.g., PND and/or rhinitis).

As described herein, compositions containing a steroid (e.g., mometasone furoate), an anticholinergic (e.g., ipratropium bromide), and an antihistamine (e.g., diphenhydramine) act synergistically and can be topically administered (e.g., in a nasal spray), at considerably lower dosage of each individual agent, to a mammal to reduce or eliminate posterior rhinorrhea. This synergistic combination increases the efficacy as well as decreases the adverse effects of component ingredients. Also described herein is a nasal cavity delivery device that can be used to bypass the anatomical nasal valve area. Bypassing this anatomical barrier allows the composition to reach the nasal cavity which is lined by ciliated epithelial cells. This nasal cavity delivery device avoids deposition of the composition in the nasal vestibule, which is lined by squamous non-ciliated epithelial cells, and does facilitate transport of the composition throughout the nasal passages.

In general, one aspect of this document features a composition including a steroid, an anticholinergic, and an antihistamine. In some embodiments, the steroid can include about 3 mg/5 mL to about 4 mg/5 mL mometasone furoate (e.g., about 3.4 mg/5 mL mometasone furoate), the anticholinergic can include about 1.5 mg/5 mL to about 2.5 mg/5 mL ipratropium bromide (e.g., about 2 mg/5 mL ipratropium bromide), and the antihistamine can include about 3 mg/5 mL to about 4 mg/5 mL diphenhydramine (e.g., about 3.4 mg/5 mL diphenhydramine). In some embodiments, the steroid can include at least 1.8 mg/5 mL mometasone furoate, the anticholinergic can include at least 1.05 mg/5 mL ipratropium bromide, and the antihistamine can include at least 1.8 mg/5 mL diphenhydramine. The composition can be in the form of a liquid or in the form of a lyophilized powder.

In another aspect, this document features a nasal spray composition containing a steroid, an anticholinergic, and an antihistamine. In some embodiments, the steroid can include about 60 μg/spray to about 75 μg/spray mometasone furoate (e.g., about 68 μg/spray mometasone furoate), the anticholinergic can include about 30 μg/spray to about 50 μg/spray ipratropium bromide (e.g., about 40 μg/spray ipratropium bromide), and the antihistamine can include about 60 μg/spray to about 75 μg/spray diphenhydramine (e.g., about 68 μg/spray diphenhydramine). In some embodiments, the steroid can include at least 35 μg/spray mometasone furoate, the anticholinergic can include at least 21 μg/spray ipratropium bromide, and the antihistamine can include at least 35 μg/spray diphenhydramine. The nasal spray composition can be in the form of a liquid or in the form of a lyophilized powder.

In another feature, this document features methods of treating chronic cough. In some aspects, the methods include, or consist essentially of, administering to a mammal having chronic cough a composition including a steroid, an anticholinergic, and an antihistamine, such that a symptom associated with chronic cough is reduced or eliminated. In some embodiments, the steroid comprises about 3 mg/5 mL to about 4 mg/5 mL mometasone furoate (e.g., about 3.4 mg/5 mL mometasone furoate), said anticholinergic comprises about 1.5 mg/5 mL to about 2.5 mg/5 mL ipratropium bromide (e.g., about 2 mg/5 mL ipratropium bromide), and said antihistamine comprises about 3 mg/5 mL to about 4 mg/5 mL diphenhydramine (e.g., about 3.4 mg/5 mL diphenhydramine). In some embodiments, the steroid includes at least 1.8 mg/5 mL mometasone furoate, the anticholinergic includes at least 1.05 mg/5 mL ipratropium bromide, and the antihistamine includes at least 1.8 mg/5 mL diphenhydramine. The administration can be a nasal administration. The nasal administration can be to a nasal cavity. The mammal can be a human. In some aspects, the methods include, or consist essentially of, administering to a mammal having chronic cough a nasal spray composition containing a steroid, an anticholinergic, and an antihistamine, such that a symptom associated with chronic cough is reduced or eliminated. In some embodiments, the steroid can include about 60 μg/spray to about 75 μg/spray mometasone furoate (e.g., about 68 μg/spray mometasone furoate), the anticholinergic can include about 30 μg/spray to about 50 μg/spray ipratropium bromide (e.g., about 40 μg/spray ipratropium bromide), and the antihistamine can include about 60 μg/spray to about 75 μg/spray diphenhydramine (e.g., about 68 μg/spray diphenhydramine). In some embodiments, the steroid can include at least 35 μg/spray mometasone furoate, the anticholinergic can include at least 21 μg/spray ipratropium bromide, and the antihistamine can include at least 35 μg/spray diphenhydramine. The mammal can be a human.

In another feature, this document features methods of treating post nasal drip. In some aspects, the methods include, or consist essentially of, administering to a mammal having post nasal drip a composition containing a steroid, an anticholinergic, and an antihistamine, such that the post nasal drip is reduced or eliminated. In some embodiments, the steroid can include about 3 mg/5 mL to about 4 mg/5 mL mometasone furoate (e.g., about 3.4 mg/5 mL mometasone furoate), the anticholinergic can include about 1.5 mg/5 mL to about 2.5 mg/5 mL ipratropium bromide (e.g., about 2 mg/5 mL ipratropium bromide), and the antihistamine can include about 3 mg/5 mL to about 4 mg/5 mL diphenhydramine (e.g., about 3.4 mg/5 mL diphenhydramine). In some embodiments, the steroid includes at least 1.8 mg/5 mL mometasone furoate, the anticholinergic includes at least 1.05 mg/5 mL ipratropium bromide, and the antihistamine includes at least 1.8 mg/5 mL diphenhydramine. The administration can be a nasal administration. The nasal administration can be to a nasal cavity. The mammal can be a human. In some aspects, the methods include, or consist essentially of, administering to a mammal having postnasal drip a nasal spray composition containing a steroid, an anticholinergic, and an antihistamine, such that the post nasal drip is reduced or eliminated. In some embodiments, the steroid can include about 60 μg/spray to about 75 μg/spray mometasone furoate (e.g., about 68 μg/spray mometasone furoate), the anticholinergic can include about 30 μg/spray to about 50 μg/spray ipratropium bromide (e.g., about 40 μg/spray ipratropium bromide), and the antihistamine can include about 60 μg/spray to about 75 μg/spray diphenhydramine (e.g., about 68 μg/spray diphenhydramine). In some embodiments, the steroid can include at least 35 μg/spray mometasone furoate, the anticholinergic can include at least 21 μg/spray ipratropium bromide, and the antihistamine can include at least 35 μg/spray diphenhydramine. The administration can be a nasal administration. The nasal administration can be to a nasal cavity. The mammal can be a human.

In another feature, this document features kits including a composition including a steroid, an anticholinergic, and an antihistamine. In some embodiments, the steroid can include about 3 mg/5 mL to about 4 mg/5 mL mometasone furoate (e.g., about 3.4 mg/5 mL mometasone furoate), the anticholinergic can include about 1.5 mg/5 mL to about 2.5 mg/5 mL ipratropium bromide (e.g., about 2 mg/5 mL ipratropium bromide), and the antihistamine can include about 3 mg/5 mL to about 4 mg/5 mL diphenhydramine (e.g., about 3.4 mg/5 mL diphenhydramine). In some embodiments, the steroid can include at least 1.8 mg/5 mL mometasone furoate, the anticholinergic can include at least 1.05 mg/5 mL ipratropium bromide, and the antihistamine can include at least 1.8 mg/5 mL diphenhydramine. The composition can be in the form of a lyophilized powder. The kit can also include a nasal cavity delivery device. The nasal cavity delivery device can include an atomizer stem. The atomizer stem can be about 1 inch to about 5 inches long.

In another feature, this document features a lyophilized powder composition containing a steroid, an anticholinergic, and an antihistamine. The steroid can include about 3 mg to about 4 mg mometasone furoate, the anticholinergic can include about 1.5 mg to about 2.5 mg ipratropium bromide, and the antihistamine can include about 3 mg to about 4 mg diphenhydramine. The lyophilized powder can be reconstituted to include about 3 mg/5 mL to about 4 mg/5 mL mometasone furoate (e.g., about 3.4 mg/5 mL mometasone furoate), about 1.5 mg/5 mL to about 2.5 mg/5 mL ipratropium bromide (e.g., about 2 mg/5 mL ipratropium bromide), and about 3 mg/5 mL to about 4 mg/5 mL diphenhydramine (e.g., about 3.4 mg/5 mL diphenhydramine).

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows an exemplary nasal cavity delivery tool including a nasal atomizer having an atomizer stem that can bypass the nasal valve and deliver a composition directly into the nasal cavity.

### DETAILED DESCRIPTION

This document provides methods and materials for using compositions containing a steroid, an anticholinergic, and an antihistamine to treat chronic cough and/or nasal congestion (e.g., chronic cough and/or nasal congestion caused by PND and/or vasodilators). For example, this document provides compositions containing a steroid (e.g., mometasone furoate), an anticholinergic (e.g., ipratropium bromide), and an antihistamine (e.g., diphenhydramine); as well as methods of using such compositions.

A composition provided herein can contain a steroid (e.g., mometasone furoate), an anticholinergic (e.g., ipratropium bromide), and an antihistamine (e.g., diphenhydramine). As described herein, a composition containing a steroid, an anticholinergic, and an antihistamine can be administered to any appropriate mammal to treat the mammal for chronic cough and/or to treat the mammal for a condition causing chronic cough. Chronic cough can include any character of the cough (e.g., barking, honking, productive vs nonproductive). Examples of conditions that can cause chronic cough include, without limitation, posterior rhinorrhea, infection (e.g., mycobacterial-, pertussis-, fungal-, and protozoal-related infections), airway disorders (e.g., asthma, nonasthmatic eosinophilic bronchitis (NAEB), bronchiectasis, atopic cough, chronic bronchitis, sarcoidosis, aspiration, and airway foreign bodies), lung parenchymal diseases (e.g., interstitial lung diseases, pulmonary hypersensitivity syndrome, and sarcoidosis), tumors (e.g., benign and malignant tumors involving the airways and parenchyma), irritation of the external auditory meatus (the Arnold reflex), upper airway cough syndrome (UACS; e.g., allergic and non-allergic rhinitis, chronic rhinosinusitis, and gastroesophageal reflux disease (GERD)), esophageal causes (e.g., GERD, laryngopharyngeal reflux, and tracheoesophageal fistula), drugs (e.g., angiotensin-converting enzyme (ACE) inhibitors, β-blockers, and vasodilators), and airway irritants (e.g., tobacco smoke, dust, fumes, and chemicals). For example, a composition containing a steroid, an anticholinergic, and an antihistamine can be administered to any appropriate mammal to reduce or eliminate PND and/or nasal congestion in the mammal.

A composition containing a steroid, an anticholinergic, and an antihistamine provided herein can include any appropriate steroid. For example, the steroid can be a glucorticosteroid. Examples of steroids that can be included within a composition provided herein include, without limitation, beclomethasone dipropionate, budesonide, ciclesonide, flunisolide, fluticasone, mometasone furoate, and triamcinolone acetonide (OTC). In some cases, the steroid can be mometasone furoate.

Any appropriate dose of a steroid provided herein can be used to formulate a composition containing a steroid, an anticholinergic, and an antihistamine provided herein. In some cases, a composition containing a steroid, an anticholinergic, and an antihistamine provided herein can contain mometasone furoate, or an equipotent dose of another steroid, in an amount from about 0.5 mg/5 mL to about 1.5 mg/5 mL (e.g., about 0.6 mg/5 mL to about 1.3 mg/5 mL, about 0.7 mg/5 mL to about 1 mg/5 mL, or about 0.8 mg/5 mL to about 1 mg/5 mL) or about 1.8 mg/5 mL to about 30 mg/5 mL (e.g., about 2.5 mg/5 mL to about 20 mg/5 mL, about 3 mg/5 mL to about 15 mg/5 mL, or about 3.4 mg/5 mL to about 10 mg/5 mL). For example, a composition containing a steroid, an anticholinergic, and an antihistamine provided herein can contain mometasone furoate, or an equipotent dose of another steroid, in an amount from about 3 mg/5 mL to about 4 mg/5 mL (e.g., about 3.4 mg/5 mL). For example, a composition containing a steroid, an anticholinergic, and an antihistamine provided herein can contain at least 1.8 mg/5 mL of mometasone furoate, or an equipotent dose of another steroid. In some cases, a composition containing a steroid, an anticholinergic, and an antihistamine provided herein can contain a steroid other than mometasone furoate in an amount from about 0.5 mg/5 mL to about 30 mg/5 mL (e.g., about 0.6 mg/5 mL to about 25 mg/5 mL, about 0.7 mg/5 mL to about 20 mg/5 mL, about 0.8 mg/5 mL to about 15 mg/5 mL, about 1 mg/5 mL to about 10 mg/5 mL, about 1.2 mg/5 mL to about 5 mg/5 mL, or about 1.5 mg/5 mL to about 3.6 mg/5 mL). For example, a composition containing a steroid, an anticholinergic, and an antihistamine provided herein can contain a steroid other than mometasone furoate in an amount from about 1.5 mg/5 mL to about 3.6 mg/5 mL (e.g., about 1.7 mg/5 mL). In some cases, a nasal spray composition containing a steroid, an anticholinergic, and an antihistamine provided herein can contain mometasone furoate, or an equipotent dose of another steroid, in an amount from about 5 μg/spray to about 30 μg/spray (e.g., about 7 μg/spray to about 25 μg/spray, about 10 μg/spray to about 20 μg/spray, or about 12 μg/spray to about 15 μg/spray) or about 35 μg/spray to about 1000 μg/spray (e.g., about 40 μg/spray to about 800 μg/spray, about 50 μg/spray to about 700 μg/spray, about 55 μg/spray to about 600 μg/spray, or about 60 μg/spray to about 500 μg/spray). For example, a nasal spray composition containing a steroid, an anticholinergic, and an antihistamine provided herein can contain mometasone furoate, or an equipotent dose of another steroid, in an amount from about 60 μg/spray to about 75 μg/spray (e.g., about 68 μg/spray). For example, a composition containing a steroid, an anticholinergic, and an antihistamine provided herein can contain at least 35 μg/spray of mometasone furoate, or an equipotent dose of another steroid. In some cases, a nasal spray composition containing a steroid, an anticholinergic, and an antihistamine provided herein can contain a steroid other than mometasone furoate in an amount from about 0.5 μg/spray to about 800 μg/spray (e.g., about 1 μg/spray to about 700 μg/spray, about 5 μg/spray to about 50 μg/spray, about 500 μg/spray to about 40 μg/spray, about 15 μg/spray to about 300 μg/spray, about 20 μg/spray to about 200 μg/spray, about 25 μg/spray to about 100 μg/spray, or about 30 μg/spray to about 70 μg/spray). For example, a nasal spray composition containing a steroid, an anticholinergic, and an antihistamine provided herein can contain a steroid other than mometasone furoate in an amount from about 30 μg/spray to about 70 μg/spray (e.g., about 34 μg/spray). In some cases, a composition containing a steroid, an anticholinergic, and an antihistamine provided herein can be formulated to deliver about 68 μg/spray of mometasone furoate, or an equipotent dose of another steroid, to a mammal (e.g., a human). For example, a nasal spray can be formulated such that a human can administer two sprays to each nostril once a day to provide about 68 μg of mometasone furoate to each nostril per day.

Exemplary doses of steroids that can be used to formulate a composition containing a steroid, an anticholinergic, and an antihistamine provided herein are shown in Table 1.

TABLE 1
Steroids.

| | Current range for | Proposed | | | | |
|---|---|---|---|---|---|---|
| | commercial products | strength | | Low range | | High range |
| Steroid | (per day) | (per day) | | (per day) | | (per day) |
| Beclomethasone | 42-336 μg | 84 | μg | 20 | μg | 680 μg |
| dipropionate | | | | | | |
| Budesonide | 32-256 μg | 64 | μg | 16 | μg | 512 μg |
| Ciclesonide | 50-800 μg | 100 | μg | 25 | μg | 1600 μg |
| Flunisolide | 25-400 μg | 50 | μg | 12.5 | μg | 800 μg |
| Fluticasone | 50-400 μg | 100 | μg | 25 | μg | 800 μg |
| Mometasone furoate | 50-800 μg | 3.4 mg/5 ml or | | 0.85 mg/5 ml or | | 800 μg |
| | | 68 μg/spray | | 17 μg | | |
| Triamcinolone | 55-220 μg | 110 | μg | 27.5 | μg | 440 μg |
| acetonide (OTC) | | | | | | |

A composition containing a steroid, an anticholinergic, and an antihistamine provided herein can include any appropriate anticholinergic. For example, the anticholinergic can be an anti-muscarinic receptor agent. Examples of anticholinergics that can be included within a composition provided herein include, without limitation, aclidinium, ipratropium bromide, tiotropium, umeclidinium, glycopyrronium bromide, glycopyrrolate, propantheline bromide, methscopolamine, oxybutynin, tolterodine, solifenacin, trospium, and darifenacin. In some cases, the anticholinergic is ipratropium bromide.

Any appropriate dose of an anticholinergic provided herein can be used to formulate a composition containing a steroid, an anticholinergic, and an antihistamine provided herein. In some cases, a composition containing a steroid, an anticholinergic, and an antihistamine provided herein can contain ipratropium bromide, or an equipotent dose of another anticholinergic, in an amount from about 0.1 mg/5 mL to about 1 mg/5 mL (e.g., about 0.2 mg/5 mL to about 0.8 mg/5 mL, about 0.3 mg/5 mL to about 0.7 mg/5 mL, or about 0.4 mg/5 mL to about 0.6 mg/5 mL) or about 1.05 mg/5 mL to about 30 mg/5 mL (e.g., about 2.5 mg/5 mL to about 20 mg/5 mL, about 3 mg/5 mL to about 15 mg/5 mL, or about 3.4 mg/5 mL to about 10 mg/5 mL). For example, a composition containing a steroid, an anticholinergic, and an antihistamine provided herein can contain ipratropium bromide, or an equipotent dose of another anticholinergic, in an amount from about 1.5 mg/5 mL to about 2.5 mg/5 mL (e.g., about 2 mg/5 mL). For example, a composition containing a steroid, an anticholinergic, and an antihistamine provided herein can contain at least 1.05 mg/5 mL of ipratropium bromide, or an equipotent dose of another anticholinergic. In some cases, a composition containing a steroid, an anticholinergic, and an antihistamine provided herein can contain an anticholinergic other than ipratropium bromide in an amount from about 0.25 mg/5 mL to about 50 mg/5 mL (e.g., about 0.3 mg/5 mL to about 25 mg/5 mL, about 0.4 mg/5 mL to about 20 mg/5 mL, about 0.5 mg/5 mL to about 15 mg/5 mL, about 0.6 mg/5 mL to about 10 mg/5 mL, about 0.7 mg/5 mL to about 5 mg/5 mL, or about 0.8 mg/5 mL to about 3 mg/5 mL). For example, a composition containing a steroid, an anticholinergic, and an antihistamine provided herein can contain an anticholinergic other than ipratropium bromide in an amount from about 0.8 mg/5 mL and about 3 mg/5 mL (e.g., about 1 mg/5 mL). In some cases, a nasal spray composition containing a steroid, an anticholinergic, and an antihistamine provided herein can contain ipratropium bromide, or an equipotent dose of another anticholinergic, in an amount from about 5 μg/spray to about 20 μg/spray (e.g., about 7 μg/spray to about 17 μg/spray, about 9 μg/spray to about 15 μg/spray, or about 10 μg/spray to about 12 μg/spray) or about 21 μg/spray to about 400 μg/spray (e.g., about 25 μg/spray to about 350 μg/spray, about 30 μg/spray to about 300 μg/spray, about 40 μg/spray to about 200 μg/spray, or about 50 μg/spray to about 100 μg/spray). For example, a nasal spray composition containing a steroid, an anticholinergic, and an antihistamine provided herein can contain ipratropium bromide, or an equipotent dose of another anticholinergic, in an amount from about 30 μg/spray to about 50 μg/spray (e.g., about 40 μg/spray). For example, a composition containing a steroid, an anticholinergic, and an antihistamine provided herein can contain at least 21 μg/spray of ipratropium bromide, or an equipotent dose of another anticholinergic. In some cases, a nasal spray composition containing a steroid, an anticholinergic, and an antihistamine provided herein can contain an anticholinergic other than ipratropium bromide in an amount from about 5 μg/spray and about 350 μg/spray (e.g., between about 8 μg/spray and about 250 μg/spray, between about 10 μg/spray and about 200 μg/spray, between about 12 μg/spray and about 100 μg/spray, between about 15 μg/spray and about 75 μg/spray, between about 17 μg/spray and about 50 μg/spray, or between about 18 μg/spray and about 45 μg/spray). For example, a nasal spray composition containing a steroid, an anticholinergic, and an antihistamine provided herein can contain an anticholinergic other than ipratropium bromide in an amount from about 18 μg/spray and about 45 μg/spray (e.g., about 20 μg/spray). In some cases, a composition containing a steroid, an anticholinergic, and an antihistamine provided herein can be formulated to deliver about 40 μg/spray of ipratropium bromide, or an equipotent dose of another anticholinergic, to a mammal (e.g., a human). For example, a nasal spray can be formulated such that a human can administer two sprays to each nostril once a day to provide about 40 μg of ipratropium bromide to each nostril per day.

Exemplary doses of anticholinergics that can be used to formulate a composition containing a steroid, an anticholinergic, and an antihistamine provided herein are shown in Table 2.

TABLE 2
Anticholinergics

| | Current range for | | Proposed | | | | | |
|---|---|---|---|---|---|---|---|---|
| | commercial products | | strength | | Low range | | High range | |
| Anticholinergic | (per day) | | (per day) | | (per day) | | (per day) | |
| Aclidinium | 200-800 | μg/day | 200 | μg | 200 | μg | 1600 | μg |
| Ipratropium bromide | 42-336 | μg | 2 mg/5 ml or 40 | | 0.5 mg/5 ml or 10 | | 336 | μg |
| | | | μg/spray | | μg/spray | | | |
| Tiotropium | 2.5-5 | μg | 2.5 | μg | 1.25 | μg/day | 20 | μg |
| Umeclidinium | 62.5 | μg | 31.25 | μg | 31.25 | μg/day | 125 | μg |
| Glycopyrronium | 4 μg/kg of body | | 50 | μg | 25 | μg/day | 100 | μg |
| bromide | weight - 400 μg as | | | | | | | |
| | an injection | | | | | | | |
| Glycopyrrolate | 40 μg-3 mg/day | | | | 20 | μg | 125 | μg |
| Propantheline Br | 7.5-75 | mg/day | 2.5 | mg | 3.75 | mg | 75 | mg |
| Methscopolamine | 2.5-20 | mg/day | 1.25 | mg | 1.25 | mg | 30 | mg |
| Oxybutynin | 5-15 | mg | 1.25 | mg | 1.25 | mg | 30 | mg |
| Tolterodine | 5-10 | mg | 1.25 | mg | 1 | mg | 20 | mg |
| Solifenacin | 5-10 | mg | 1.25 | mg | 1 | mg | 20 | mg |
| Trospium | 20-40 | mg | 5 | mg | 2.5 | ng | 80 | mg |
| Darifenacin | 7.5-15 | mg | 2.5 | mg | 1.25 | mg | 30 | mg |

A composition containing a steroid, an anticholinergic, and an antihistamine provided herein can include any appropriate antihistamine. For example, the antihistamine can block the histamine receptor subtype H₁. The antihistamine can be a first generation antihistamine, second generation antihistamine, or third generation antihistamine. Examples of antihistamines that can be included within a composition provided herein include, without limitation, azelastine HCl, olopatadine HCl, brompheniramine, chlorpheniramine, dexbrompheniramine, dexchlorpheniramine, triprolidine, carbinoxamine, clemastine, diphenhydramine HCl, doxylamine, promethazine, chlorcyclizine, hydroxyzine, cyproheptadine, cetirizine, levocetirizine, desloratadine, fexofenadine, and loratadine. In some cases, the antihistamine is diphenhydramine.

Any appropriate dose of an antihistamine provided herein can be used to formulate a composition containing a steroid, an anticholinergic, and an antihistamine provided herein. Antihistamines (e.g. diphenhydramine) cause sedation in large doses. The antihistamine can be a low, non-sedative dose of antihistamine. In some cases, a composition containing a steroid, an anticholinergic, and an antihistamine provided herein can contain diphenydramine, or an equipotent dose of another antihistamine, in an amount from about 0.5 mg/5 mL to about 1.5 mg/5 mL (e.g., about 0.6 mg/5 mL to about 1.3 mg/5 mL, about 0.7 mg/5 mL to about 1 mg/5 mL, or about 0.8 mg/5 mL to about 1 mg/5 mL) or about 1.8 mg/5 mL to about 30 mg/5 mL (e.g., about 2.5 mg/5 mL to about 20 mg/5 mL, about 3 mg/5 mL to about 15 mg/5 mL, or about 3.4 mg/5 mL to about 10 mg/5 mL). For example, a composition containing a steroid, an anticholinergic, and an antihistamine provided herein can contain diphenydramine, or an equipotent dose of another antihistamine, in an amount from about 3 mg/5 mL to about 4 mg/5 mL (e.g., about 3.4 mg/5 mL). For example, a composition containing a steroid, an anticholinergic, and an antihistamine provided herein can contain at least 1.8 mg/5 mL of diphenydramine, or an equipotent dose of another antihistamine. In some cases, a composition containing a steroid, an anticholinergic, and an antihistamine provided herein can contain an antihistamine other than diphenhydramine in an amount from about 0.5 mg/5 mL to about 30 mg/5 mL (e.g., about 0.6 mg/5 mL to about 25 mg/5 mL, about 0.7 mg/5 mL to about 20 mg/5 mL, about 0.8 mg/5 mL to about 15 mg/5 mL, about 1 mg/5 mL to about 10 mg/5 mL, about 1.2 mg/5 mL to about 5 mg/5 mL, or about 1.5 mg/5 mL to about 3.6 mg/5 mL). For example, a composition containing a steroid, an anticholinergic, and an antihistamine provided herein can contain an antihistamine other than diphenhydramine in an amount from about 1.5 mg/5 mL and about 2 mg/5 mL (e.g., about 1.7 mg/5 mL). In some cases, a nasal spray composition containing a steroid, an anticholinergic, and an antihistamine provided herein can contain diphenhydramine, or an equipotent dose of another antihistamine, in an amount from about 5 μg/spray to about 30 μg/spray (e.g., about 7 μg/spray to about 25 μg/spray, about 10 μg/spray to about 20 μg/spray, or about 12 μg/spray to about 15 μg/spray) or about 35 μg/spray to about 1000 μg/spray (e.g., about 40 μg/spray to about 800 μg/spray, about 50 μg/spray to about 700 μg/spray, about 55 μg/spray to about 600 μg/spray, or about 60 μg/spray to about 500 μg/spray). For example, a nasal spray composition containing a steroid, an anticholinergic, and an antihistamine provided herein can contain diphenhydramine, or an equipotent dose of another antihistamine, in an amount from about 60 μg/spray to about 75 μg/spray (e.g., about 68 μg/spray). For example, a composition containing a steroid, an anticholinergic, and an antihistamine provided herein can contain at least 35 μg/spray of diphenydramine, or an equipotent dose of another antihistamine. In some cases, a nasal spray composition containing a steroid, an anticholinergic, and an antihistamine provided herein can contain an antihistamine other than diphenhydramine in an amount from about 0.5 μg/spray and about 800 μg/spray (e.g., between about 1 μg/spray and about 700 μg/spray, between about 5 μg/spray and about 50 μg/spray, between about 500 μg/spray and about 40 μg/spray, between about 15 μg/spray and about 300 μg/spray, between about 20 μg/spray and about 200 μg/spray, between about 25 μg/spray and about 100 μg/spray, or between about 30 μg/spray and about 70 μg/spray). For example, a nasal spray composition containing a steroid, an anticholinergic, and an antihistamine provided herein can contain an antihistamine other than diphenhydramine in an amount from about 30 μg/spray to about 40 μg/spray (e.g., about 34 μg/spray). In some cases, a composition containing a steroid, an anticholinergic, and an antihistamine provided herein can be formulated to deliver about 68 μg/spray of diphenhydramine, or an equipotent dose of another antihistamine, to a mammal (e.g., a human). For example, a nasal spray can be formulated such that a human can administer two sprays to each nostril once a day to provide about 68 μg of diphenhydramine to each nostril per day.

Exemplary doses of antihistamines that can be used to formulate a composition containing a steroid, an anticholinergic, and an antihistamine provided herein are shown in Table 3.

TABLE 3
Antihistamines

| | | | | |
|---|---|---|---|---|
| | Current range for | Proposed | | |
| | commercial products | strength | Low range | High range |
| Antihistamine | (Per day) | (per day) | (per day) | (per day) |

1^{st} generation

| Azelastine HCl | 137-822 | μg/day | 137 μg | 69 | μg | 1644 | μg |
|---|---|---|---|---|---|---|---|
| Olopatadine HCl | 1330-2660 | μg/day | 1330 μg | 665 | μg | 5320 | μg |
| | (0.1%-0.2% | | (0.1%/spray) | (0.05%/spray) | | (0.4%/spray) | |
| | solution/spray) | | | | | | |
| Brompheniramine | 4-24 mg/day | | 0.4 mg/5 ml | 2 | μg/spray | 4 | μg/spray |
| | orally | | | | | | |
| Chlorpheniramine | 24 | mg/day orally | 2.4 mg/5 ml | 500 | μg/spray | 1000 | μg/spray |
| | | | | | | 48 | mg (oral) |
| Dexbrompheniramine | 12 | mg/day orally | 1.2 mg/5 ml | 24 | μg/spray | 48 | μg/spray |
| | | | | | | 48 | mg (oral) |
| Dexchlorpheniramine | 12 | mg/day orally | 1.2 mg/5 ml | 24 | μg/spray | 48 | μg/spray |
| | | | | | | 48 | mg (oral) |
| Triprolidine | 15 | mg/day orally | 1.5 mg/5 ml | 300 | μg/spray | 600 | μg/spray |
| Carbinoxamine | 16-32 mg/day | | 1.6 mg/5 ml | 32 | μg/spray | 64 | μg/spray |
| | orally | | | | | | |
| Clemastine | 2 | mg/day orally | 0.2 mg/5 ml | 4 | μg/spray | 8 | μg/spray |

| Diphenhydramine HCl | 12.5-300 mg/day | 3.4 mg/5 ml or | 1.7 mg/5 ml or | | 300 | mg |
|---|---|---|---|---|---|---|
| | orally | 69.2 μg/spray | 34 μg/spray | | | |
| Doxylamine | 30-60 mg/day | 3 mg/5 ml | 60 | μg/spray | 120 | μg |
| | orally | | | | | |
| Promethazine | 6.5-100 mg/day | 0.65 mg/5 ml | 13 | μg/spray | 100 | mg |
| | orally | | | | | |
| Chlorcyclizine | 75-100 mg/day | 7.5 mg/5 ml | 300 | μg/spray | 6 | mg |
| | orally | | | | | |
| Hydroxyzine | 75-100 mg/day | 7.5 mg/5 ml | 300 | μg/spray | 6 | mg |
| | orally | | | | | |
| Cyproheptadine | 12-60 mg/day | 1.2 mg/5 ml | 24 6 | μg/spray | 240 | μg |
| | orally | | | | | |

2^{nd} generation

| Cetirizine | 2.5-10 mg/day | | 0.25 mg/5 ml | 5 | μg/spray | 100 | μg |
|---|---|---|---|---|---|---|---|
| | orally | | | | | | |
| Levocetirizine | 5 | mg/day orally | 0.5 mg/5 ml | 10 | μg/spray | 200 | μg |
| Desloratadine | 5 | mg/day orally | 0.5 mg/5 ml | 10 | μg/spray | 200 | μg |
| Fexofenadine | 120 | mg/day orally | 12 mg/5 ml | 240 | μg/spray | 4800 | μg |

| | | | | | | |
|---|---|---|---|---|---|---|
| Loratadine | 5-10 mg/day | 0.5 mg/5 ml | 10 | μg/spray | 200 | μg |
| | orally | | | | | |

In some cases, a composition containing a steroid, an anticholinergic, and an antihistamine can contain mometasone furoate in an amount from about 3 mg/5 mL to about 4 mg/5 mL (e.g., about 3.4 mg/5 mL), ipratropium bromide in an amount from about 1.5 mg/5 mL to about 2.5 mg/5 mL (e.g., about 2 mg/5 mL), and diphenhydramine in an amount from about 3 mg/5 mL to about 4 mg/5 mL (e.g., about 3.4 mg/5 mL).

In some cases, a nasal spray composition containing a steroid, an anticholinergic, and an antihistamine provided herein can contain mometasone furoate in an amount from about 60 μg/spray to about 75 μg/spray (e.g., about 68 μg/spray), ipratropium bromide in an amount from about 30 μg/spray to about 50 μg/spray (e.g., about 40 μg/spray), and diphenhydramine in an amount from about 60 μg/spray to about 75 μg/spray (e.g., about 68 μg/spray).

A composition containing a steroid, an anticholinergic, and an antihistamine can contain other appropriate ingredients. Examples of other ingredients include, without limitation, decongestants, inhaled asthma drugs (e.g., bronchodilators and inhaled corticosteroids), antibiotics, acid blockers, cough suppressants, topical anaesthetics (e.g., lidocaine, bupivacaine), gabapentin, pregabalin, duloxetine, venlafaxine, and amitriptyline. In some cases, a composition containing a steroid, an anticholinergic, and an antihistamine can be formulated together with one or more additional ingredients (e.g., a decongestant) to form a single composition. In some cases, additional ingredients (e.g., a decongestant) can be provided to a mammal in a separate composition; one containing a steroid, an anticholinergic, and an antihistamine, and one containing, for example, the decongestant.

In some cases, a composition containing a steroid, an anticholinergic, and an antihistamine can be formulated as a pharmaceutical composition. For example, a composition containing a steroid, an anticholinergic, and an antihistamine provided herein can contain a pharmaceutically acceptable carrier for administration to a mammal, including, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other known carriers for topical administration.

An acceptable aqueous vehicle can be, for example, any liquid solution that is capable of dissolving a composition containing a steroid, an anticholinergic, and an antihistamine provided herein and is not toxic to the particular individual receiving the composition. Examples of acceptable aqueous vehicles include, without limitation, saline, water, and acetic acid. Typically, acceptable aqueous vehicles are sterile. An acceptable solid vehicle can be formulated such that compositions composition containing a steroid, an anticholinergic, and an antihistamine provided herein are suitable for topical administration. The dose supplied can vary since an effective amount can be reached by either one or multiple administrations. Any appropriate pharmaceutically acceptable material such as gelatin and cellulose derivatives can be used as an acceptable solid vehicle. In addition, an acceptable solid vehicle can be a solid carrier including, without limitation, starch, sugar, or bentonite. Further, a topical formulation of a composition containing a steroid, an anticholinergic, and an antihistamine can follow conventional procedures that employ solid carriers, lubricants, and the like. In some cases, a composition containing a steroid, an anticholinergic, and an antihistamine can be formulated for nasal topical application.

Any appropriate method can be used to formulate a pharmaceutical composition provided herein (e.g., a pharmaceutical composition containing a steroid, an anticholinergic, and an antihistamine provided herein). For example, common formulation mixing and preparation techniques can be used to make a composition having the components described herein. In addition, the compositions provided herein can be in any appropriate form. For example, a composition provided herein can be in the form of a solid, liquid, and/or aerosol including, without limitation, powders, crystalline substances, gels, pastes, ointments, salves, creams, solutions, suspensions, partial liquids, sprays, nebulae, mists, atomized vapors, tinctures, pills, capsules, tablets, and gelcaps. In some embodiments, composition containing a steroid, an anticholinergic, and an antihistamine provided herein can be prepared for topical administration by mixing the components with one or more of the following: a filler, a binder, a disintegrator, a lubricant, and a coloring agent. Lactose, corn starch, sucrose, glucose, trehalose, glycine, mannitol, sorbitol, crystalline cellulose, silicon dioxide, or the like can be used as the filler. Polyvinyl alcohol, polyvinyl ether, ethyl cellulose, methyl cellulose, acacia, tragacanth, gelatin, shellac, hydroxypropyl cellulose, hydroxypropylmethyl cellulose, calcium citrate, dextrin, or pectin can be used as the binder. Magnesium stearate, talc, polyethylene glycol, silica, or hardened plant oil can be used as the lubricant. A pharmaceutically acceptable coloring agent can be used as the coloring agent. Cocoa powder, mentha water, aromatic acid, mentha oil, borneol, or powdered cinnamon bark also can be added.

This document also provides methods for using composition containing a steroid, an anticholinergic, and an antihistamine provided herein. For example, compositions provided herein can be used to treat a mammal having chronic cough, to treat a condition causing a mammal to have chronic cough, and/or to reduce or eliminate PND in a mammal. For example, compositions provided herein can be used to treat a mammal having nasal congestion (e.g., to reduce or eliminate nasal in a mammal).

Methods for treating a mammal having chronic cough, methods for treating a mammal having a condition causing chronic cough, and/or methods for reducing or eliminating PND can include administering to the mammal a composition containing a steroid, an anticholinergic, and an antihistamine provided herein. Methods for treating a mammal having chronic cough and/or methods for treating a mammal having a condition causing chronic cough (e.g., PND) can be effective to reduce or eliminate one or more conditions causing chronic cough. Examples of conditions causing chronic cough that can be treated using the compositions containing a steroid, an anticholinergic, and an antihistamine provided herein include, without limitation, posterior rhinorrhea, infection (e.g., mycobacterial-, pertussis-, fungal-, and protozoal-related infections), airway disorders (e.g., asthma, NAEB), bronchiectasis, atopic cough, chronic bronchitis, sarcoidosis, aspiration, and airway foreign bodies), lung parenchymal diseases (e.g., interstitial lung diseases, pulmonary hypersensitivity syndrome, and sarcoidosis), tumors (e.g., benign and malignant tumors involving the airways and parenchyma), irritation of the external auditory meatus (the Arnold reflex), UACS (e.g., allergic and non-allergic rhinitis, chronic rhinosinusitis, and GERD), esophageal causes (e.g., GERD, laryngopharyngeal reflux, and tracheoesophageal fistula), drugs (e.g., ACE inhibitors and β-blockers), airway irritants (e.g., tobacco smoke, dust, fumes, and chemicals). Methods for treating a mammal having chronic cough and/or methods for treating a mammal having a condition causing chronic cough (e.g., PND) can be effective to reduce or eliminate one or more symptoms associated with chronic cough. Examples of symptoms associated with chronic cough include, without limitation, cough, anterior rhinorrhea (runny nose), posterior rhinorrhea, nasal pruritus (nasal itching), nasal congestion, sneezing, anxiety, frustration, and depression.

Methods for treating a mammal having nasal congestion can include administering to the mammal a composition containing a steroid, an anticholinergic, and an antihistamine provided herein. Methods for treating a mammal having nasal congestion can be effective to reduce or eliminate the nasal congestion. In some cases, the nasal congestion can be caused by a vasodilator. Examples of vasodilators include, without limitation, phosphodiesterase type 5 inhibitor (PDE5) inhibitors (e.g., sildenafil, avanafil, lodenafil, mirodenafil, tadalafil, vardenafil, udenafil, zaprinast, icariin and its synthetic derivatives, benzamidenafil, and dasantafil), and prostacyclin and its analogs (e.g., iloprost and cisaprost). For example, a mammal having pulmonary arterial hypertension can take a vasodilator that causes nasal congestion.

Methods for treating a mammal having chronic cough and/or nasal congestion can include identifying the mammal as having chronic cough and/or nasal congestion. Examples of methods for identifying the mammal as having chronic cough and/or a condition causing chronic cough include, without limitation, imaging tests (e.g., X-rays, computerized tomography (CT) scans, and chest radiography), lung function tests (e.g., spirometry and exhaled nitric oxide (NO) measurement), lab tests, and/or scope tests (e.g., flexible rhinolaryngoscopy). Methods for reducing or eliminating PND in a mammal can include identifying the mammal as having PND. Once identified as having chronic cough and/or having a condition causing chronic cough (e.g., PND), the mammal can be administered or instructed to self-administer a composition containing a steroid, an anticholinergic, and an antihistamine provided herein.

In some cases, methods for treating a mammal having chronic cough, methods for treating a mammal having nasal congestion, and/or methods for treating a mammal having a condition causing chronic cough can include additional treatment such as decongestants, inhaled asthma drugs (e.g., bronchodilators and inhaled corticosteroids), antibiotics, Histamine H2 blockers, cough suppressants, topical anaesthetics (e.g., lidocaine, bupivacaine), gabapentin, pregabalin, duloxetine, venlafaxine, amitriptyline, proton pump inhibitors, and/or baclofen.

Compositions containing a steroid, an anticholinergic, and an antihistamine provided herein can be administered to any mammal (e.g., rat, mouse, dog, cat, horse, cow, goat, pig, monkey, or human). In addition, any route of administration (e.g., topical or systemic) can be used to administer a composition provided herein to a mammal. In some cases, administration of a composition provided herein can be nasal administration (e.g., nasal sprays or nasal drops).

Any appropriate dose of a composition containing a steroid, an anticholinergic, and an antihistamine provided herein can be used to formulate pharmaceuticals provided herein (e.g., a pharmaceutical including a composition containing a steroid, an anticholinergic, and an antihistamine). For example, a composition provided herein can be formulated to contain between about 1.5 mg/5 mL and about 3.6 mg/5 mL (e.g., about 1.7 mg/5 mL or about 3.4 mg/5 mL) of steroid (e.g., mometasone furoate), about between about 0.8 mg/5 mL and about 3 mg/5 mL (e.g., about 1 mg/5 mL or about 2 mg/5 mL) of anticholinergic (e.g., ipratropium bromide), and between about 1.5 mg/5 mL and about 3.6 mg/5 mL (e.g., about 1.7 mg/5 mL or about 3.4 mg/5 mL) of antihistamine (e.g., diphenhydramine). For example, a nasal spray composition provided herein can be formulated to deliver about 34 μg of steroid (e.g., mometasone furoate), about 20 μg of anticholinergic (e.g., ipratropium bromide), and about 34 μg of antihistamine (e.g., diphenhydramine), to a mammal (e.g., a human) per spray. Various factors can influence the actual amount used for a particular application. For example, the frequency of administration, duration of treatment, combination of other agents, site of administration, stage of disease (if present), and the anatomical configuration of the treated area may require an increase or decrease in the actual amount administered.

The frequency of administration of compositions containing a steroid, an anticholinergic, and an antihistamine provided herein can be any frequency. For example, the frequency of administration can be from about four times a day to about once a month, or more specifically, from about twice a day to about once a week. In some cases, a composition provided herein can be formulated into a nasal spray composition containing about 68 μg/spray of steroid (e.g., mometasone furoate), about 40 μg/spray of anticholinergic (e.g., ipratropium bromide), and about 68 μg/spray of antihistamine (e.g., diphenhydramine), that a mammal (e.g., a human) can administer (e.g., spray) once a day to provide about 68 μg of steroid (e.g., mometasone furoate), about 40 μg of anticholinergic (e.g., ipratropium bromide), and about 68 μg of antihistamine (e.g., diphenhydramine) per day. In some cases, a composition provided herein can be formulated into a nasal spray composition containing about 34 μg/spray of steroid (e.g., mometasone furoate), about 20 μg/spray of anticholinergic (e.g., ipratropium bromide), and about 34 μg/spray of antihistamine (e.g., diphenhydramine), that a mammal (e.g., a human) can administer (e.g., spray) twice a day to provide about 68 μg of steroid (e.g., mometasone furoate), about 40 μg of anticholinergic (e.g., ipratropium bromide), and about 68 μg of antihistamine (e.g., diphenhydramine) per day. In addition, the frequency of administration can remain constant or can be variable during the duration of treatment. As with the amount administered, various factors can influence the actual frequency of administration used for a particular application. For example, the amount (dose), duration of treatment, combination of agents, site of administration, stage of disease (if present), presence or absence of nasal polyps, acute and chronic sinusitis, postsurgical nasosinus anatomy, and the anatomical configuration of the treated area may require an increase or decrease in administration frequency.

The duration of administration of a composition containing a steroid, an anticholinergic, and an antihistamine provided herein can be any duration. For example, a duration of administration of compositions provided herein can be longer than a week, month, three months, six months, nine months, a year, two years, or three years. In some cases, an effective duration can be any duration that reduces, prevents, or eliminates PND upon administration to a mammal without producing significant toxicity to the mammal. Such an effective duration can vary from several days to several weeks, months, or years. In general, an effective duration for the treatment of an acute disease can range in duration from several days to several months. Once administration of compositions containing a steroid, an anticholinergic, and an antihistamine provided herein is stopped, however, symptoms may return. In such cases, an effective duration for the prevention of certain conditions can last for as long as the individual is alive. Multiple factors can influence the actual duration used for a particular treatment or prevention regimen. For example, an effective duration can vary with the frequency of administration, the amount administered, combination of multiple agents, site of administration, state of disease (if present), and anatomical configuration of the treated area.

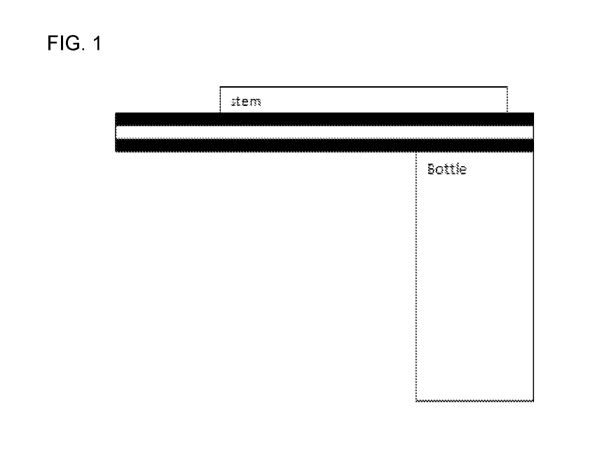

This document also provides materials for administering a composition containing a steroid, an anticholinergic, and an antihistamine provided herein. For example, a nasal cavity delivery device can be used to administer compositions provided herein to the nasal cavity of a mammal having chronic cough and/or a condition causing a mammal to have chronic cough (e.g., PND). A nasal cavity delivery device includes a bottle, an atomizer, and a stem. In some cases, a nasal cavity delivery device includes the atomizer and the stem together as an atomizer stem. An exemplary nasal cavity delivery device is shown in FIG. 1. The bottle can be in the form of a squeeze bottle, syringe tube, dropper, or syringe attached to a mucosal atomizer device. The bottle can contain the composition containing a steroid, an anticholinergic, and an antihistamine provided herein in any appropriate form. For example, the bottle can contain a powdered composition (e.g., a lyophilized composition) or a liquid composition. The atomizer stem can deliver a powdered composition (e.g., a lyophilized composition) or a liquid composition. The atomizer stem can be long enough to bypass an anatomic barrier (e.g., the nasal valve) and deliver the composition into the nasal cavity. For example, the atomizer stem can be from about 1 inch to about 5 inches long (e.g., about 1.5 inches to about 4.5 inches, about 1.75 inches to about 4 inches, or about 2 inches to about 3 inches). In some cases, the atomizer stem can be about 2.125 inches long. The nasal cavity delivery device can administer the composition provided herein by any appropriate means. For example, in cases where the bottle is a squeeze bottle, the nasal cavity delivery device can administer the composition by squeezing the bottle; and in cases where the bottle is a syringe, the nasal cavity delivery device can administer the composition by depressing the syringe plunger.

Also provided herein are kits. Typically, a kit includes a composition containing a steroid, an anticholinergic, and an antihistamine provided herein. In some cases, the composition containing a steroid, an anticholinergic, and an antihistamine is a lyophilized composition. A kit can also include a device (e.g., a nasal cavity delivery device provided herein) to deliver the composition containing a steroid, an anticholinergic, and an antihistamine provided herein. In some cases, the device delivers the composition beyond the nasal valve and into the nasal cavity. A kit can also include directions for use of the kit. For example, the kit can include instructions for reconstituting the lyophilized composition in any appropriate solute (e.g., a physiologically acceptable aqueous vehicle such as saline). For example, the kit can include instructions for reconstituting the lyophilized composition to provide doses for 4 to 6 days. For example, the kit can include instructions for administering the composition.

The invention will be further described in the following examples, which do not limit the scope of the invention described in the claims.

### EXAMPLES

### Example 1: Treatment of Postnasal Drip

A composition including mometasone furoate (1.7 mg/5 mL), ipratropium bromide (1 mg/5 mL), and diphenhydramine (1.7 mg/5 mL) was prepared.

A specially configured nasal atomizer having a large aerosol plume and a three inch stem was used to bypass the nasal valve area and deliver medication into the nasal cavity. Two sprays were administered to each nostril daily. Patient responses were evaluated after 4 to 6 weeks.

Diphenhydramine at 34 μg/spray was effective in arresting postnasal drip in combination with an anticholinergic and a glucocorticosteroid. These results suggest that there is synergy among three groups of medications when used together topically.

34 micrograms/spray (or 69 μg daily) to each nostril is 5000 times less than the lowest dose (12.5 milligrams) of oral diphenhydramine. With 15,000 units dispensed, sedation has not been an issue. These results suggest that first generation antihistamine (e.g., diphenhydramine) can be effective at low doses with good patient tolerance.

### Example 2: Compositions Including a Steroid, an Anticholinergic, and an Antihistamine

A composition including mometasone furoate (3.4 mg/5 mL), ipratropium bromide (2 mg/5 mL), and diphenhydramine (3.4 mg/5 mL) is prepared.

A specially configured nasal atomizer having a large aerosol plume and a three inch stem will be used to bypass the nasal valve area and deliver medication into the nasal cavity.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A composition comprising a steroid, an anticholinergic, and an antihistamine, wherein said steroid comprises about 3 mg/5 mL to about 4 mg/5 mL mometasone furoate, said anticholinergic comprises about 1.5 mg/5 mL to about 2.5 mg/5 mL ipratropium bromide, and said antihistamine comprises about 3 mg/5 mL to about 4 mg/5 mL diphenhydramine.

2. The composition of claim 1, wherein said steroid comprises about 3.4 mg/5 mL mometasone furoate.

3. The composition of claim 1, wherein said anticholinergic comprises about 2 mg/5 mL ipratropium bromide.

4. The composition of claim 1, wherein said antihistamine comprises about 3.4 mg/5 mL diphenhydramine.

5. The composition of claim 1, wherein said composition is in the form of a liquid.

6. The composition of claim 1, wherein said composition is in the form of a lyophilized powder.

7. A nasal spray composition containing a steroid, an anticholinergic, and an antihistamine, wherein said steroid comprises about 60 μg/spray to about 75 μg/spray mometasone furoate, said anticholinergic comprises about 30 μg/spray to about 50 μg/spray ipratropium bromide, and said antihistamine comprises about 60 μg/spray to about 75 μg/spray diphenhydramine.

8. The nasal spray composition of claim 7, wherein said steroid comprises about 68 μg/spray mometasone furoate.

9. The nasal spray composition of claim 7, wherein said anticholinergic comprises about 40 μg/spray ipratropium bromide.

10. The nasal spray composition of claim 7, wherein said antihistamine comprises about 68 μg/spray diphenhydramine.

11. The nasal spray composition of claim 7, wherein said composition is in the form of a liquid.

12. The nasal spray composition of claim 7, wherein said composition is in the form of a lyophilized powder.

13.-32. (canceled)

33. A kit comprising a composition comprising a steroid, an anticholinergic, and an antihistamine, wherein said steroid comprises about 3 mg/5 mL to about 4 mg/5 mL mometasone furoate, said anticholinergic comprises about 1.5 mg/5 mL to about 2.5 mg/5 mL ipratropium bromide, and said antihistamine comprises about 3 mg/5 mL to about 4 mg/5 mL diphenhydramine.

34. The kit of claim 33, wherein said steroid comprises about 3.4 mg/5 mL mometasone furoate.

35. The kit of claim 33, wherein said anticholinergic comprises about 2 mg/5 mL ipratropium bromide.

36. The kit of claim 33, wherein said antihistamine comprises about 3.4 mg/5 mL diphenhydramine.

37. The kit of claim 33, wherein said composition is in the form of a lyophilized powder.

38. The kit of claim 33, said kit further comprising a nasal cavity delivery device.

39. The kit of claim 38, wherein said nasal cavity delivery device comprises an atomizer stem.

40. The kit of claim 39, wherein said atomizer stem is about 1 inch to about 5 inches long.

41.-68. (canceled)
